(19) United States
(12) Patent Application Publication
(10) Pub. No.: US 2019/0017013 A1
(43) Pub. Date: Jan. 17, 2019

(54) MECHANICAL LYSIS APPARATUS AND METHOD

(71) Applicant: MBio Diagnostics, Inc., Boulder, CO (US)

(72) Inventors: Sarah R. Bickman, Boulder, CO (US); Keagan B. Rowley, Boulder, CO (US); Kevin Moll, Boulder, CO (US); Michael J. Lochhead, Boulder, CO (US)

(21) Appl. No.: 16/034,926

(22) Filed: Jul. 13, 2018

Related U.S. Application Data
(60) Provisional application No. 62/532,644, filed on Jul. 14, 2017.
(60) Provisional application No. 62/694,861, filed on Jul. 6, 2018.

(51) Int. Cl. C12M 1/00; C12N 1/06

(52) U.S. Cl. CPC C12M 47/06; G01N 1/286; C12N 1/066

(57) ABSTRACT

A mechanical lysis method includes (a) spinning a mixing head at a spin rate in a liquid sample, containing cells and beads, such that the mixing head cooperates with the beads to lyse the cells, wherein the spin rate exceeds a threshold rate associated with a predefined lysis efficiency, and (b) preventing the mixing head from spinning if the spin rate cannot be maintained above the threshold rate. A lysis apparatus includes (a) a receptacle for holding a container containing a liquid sample with cells and beads, (b) a mixing head configured to spin in the liquid sample, (c) a motor configured to spin the mixing head such that the mixing head cooperates with the beads to lyse the cells, and (d) a switch configured to prevent spinning of the mixing head at a spin rate lower than a threshold rate associated with a predefined lysis efficiency.

### CROSS-REFERENCE TO RELATED APPLICATIONS

The present application claims the benefit of priority from U.S. Provisional Application Ser. No. 62/532,644 filed Jul. 14, 2017, and from U.S. Provisional Application Ser. No. 62/694,861, filed Jul. 6, 2018, both of which are incorporated herein by reference in their entireties.

### U.S. GOVERNMENT RIGHTS

This invention was made with Government support under grant number 1621951 awarded by the National Science Foundation. The Government has certain rights in the invention.

### BACKGROUND

Cell disruption, cell lysis, and tissue lysis are necessary for many biological measurements. For example, detection of a certain cell type, such as a certain bacteria, may rely on detection of protein that resides inside the bacterial cell and is detectable only if released from the bacterial cell. A wide range of lysis methods exist including chemical methods, repeated freezing and thawing cells, sonication, and bead beating. There are significant advantages to methods that can be done quickly at the point of sample collection as these methods enable on-the-spot testing. However, the requirement of such testing precludes many typical methods, such as repeated freezing and thawing, that would either take too long or require equipment that is typically not available at the point of sample collection. Although chemical cell lysis may be performed quickly and without excessive equipment, chemical lysis dilutes the sample and introduce chemicals, both of which can adversely affect subsequent reactions necessary to perform assays.

### SUMMARY

In an embodiment, a mechanical lysis method includes spinning a mixing head at a spin rate in a liquid sample, containing cells and beads, such that the mixing head cooperates with the beads to lyse the cells. The spin rate exceeds a threshold rate associated with a predefined lysis efficiency. The method further includes preventing the mixing head from spinning if the spin rate cannot be maintained above the threshold rate.

In an embodiment, a lysis apparatus includes a receptacle for holding a container containing a liquid sample with cells and beads. The lysis apparatus further includes a mixing head configured to spin in the liquid sample, and a motor configured to spin the mixing head such that the mixing head cooperates with the beads to lyse the cells. In addition, the lysis apparatus includes a switch configured to prevent spinning of the mixing head at a spin rate lower than a threshold rate associated with a predefined lysis efficiency.

### BRIEF DESCRIPTION OF THE DRAWINGS

FIG. 1 illustrates a mechanical lysis method that achieves lysis of cells by rapid mixing of a liquid sample that contains both the cells and beads, according to an embodiment.

FIG. 2 is a block diagram of a spin-rate controlled mechanical lysis apparatus, according to an embodiment.

FIG. 3 is a block diagram of a spin-rate controlled mechanical lysis apparatus having a switch between a motor actuating the mixing head and a power supply powering the motor, according to an embodiment.

FIG. 4 is a block diagram of a spin-rate controlled mechanical lysis apparatus having a monitoring circuit that monitors the level of electric power provided by a motor actuating the mixing head, according to an embodiment.

FIG. 5 is a block diagram of a spin-rate controlled mechanical lysis apparatus having a feedback circuit that adjusts the spin rate of the mixing head based upon an electric power level measurement, according to an embodiment.

FIG. 6 is a block diagram of a spin-rate controlled mechanical lysis apparatus configured to monitor the spin rate of the mixing head, according to an embodiment.

FIG. 7 is a block diagram of a spin-rate controlled mechanical lysis apparatus configured with a feedback circuit that adjusts the spin rate of the mixing head based upon a spin rate measurement, according to an embodiment.

FIG. 8 is a side view of one structural configuration of a mechanical lysis apparatus, according to an embodiment.

FIG. 9 is a side view of another structural configuration of a mechanical lysis apparatus, according to an embodiment.

FIG. 10 illustrates a mixing head that includes two wire loops, according to an embodiment.

FIG. 11 illustrates a mixing head that includes a helical coil, according to an embodiment.

FIG. 12 illustrates a mixing head with a plurality of blades, according to an embodiment.

FIG. 13 is a block diagram of one spin-rate controlled mechanical lysis apparatus that utilizes magnetic coupling between a mixing head and a motor actuating the mixing head, according to an embodiment.

FIG. 14 illustrates one mechanical lysis method, according to an embodiment.

FIG. 15 illustrates a method for preventing the mixing head in a mechanical lysis apparatus from spinning at a rate that is below a threshold rate associated with a desired lysis efficiency, according to an embodiment.

FIG. 16 shows example data achieved in mechanical lysis using a helical-coil mixing head.

FIG. 17 shows example data achieved in mechanical lysis using a wire-loop mixing head.

FIG. 18 shows more example data achieved in mechanical lysis using a helical-coil mixing head.

### DETAILED DESCRIPTION OF THE EMBODIMENTS

Mechanical lysis using bead beating may be performed with portable bead beaters suitable for use in the field. U.S. Pat. No. 9,150,826 (Isely et al.) discloses a handheld shaking device that shakes a vial containing beads and a sample to be lysed. International Patent Application WO/2010/151705 (Irvine et al.) discloses a small chamber containing a plurality of beads through which the sample is passed. Recently, the use of a commercially available milk frother as a portable lysis device has been demonstrated by Devlin et al. (Devlin S, Meneely J P, Greer B, Greef C, Lochhead M J, Elliott C T, “Next generation planar waveguide detection of microcystins in freshwater and cyanobacterial extracts, utilising a novel lysis method for portable sample preparation and analysis”, Anal Chim Acta. 2013; 769:108-13). In this demonstration, the milk frother was immersed in a sample and actuated to lyse the cells without use of beads. The frother used in the Devlin et al. demonstration is similar to those described in U.S. Pat. No. 6,764,704 (Schub) and U.S. Pat. No. 6,558,035 (Lane).

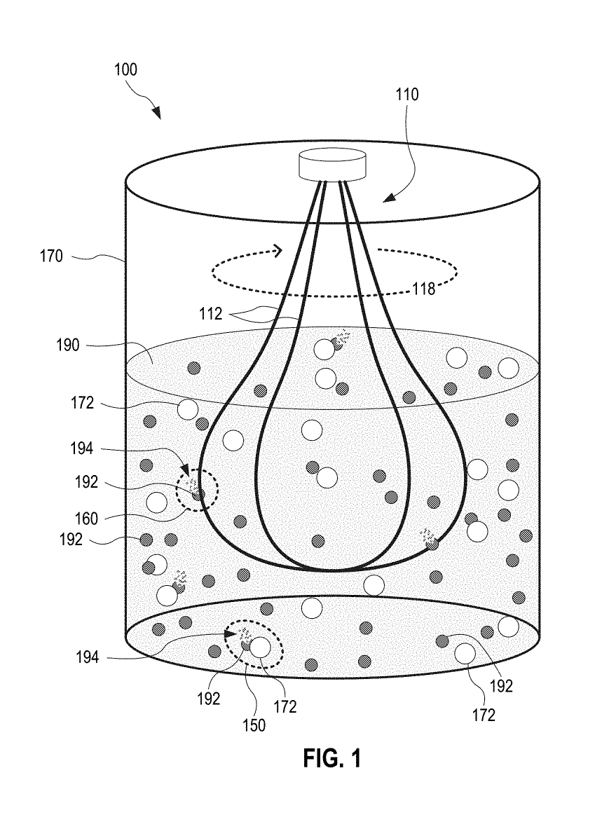

FIG. 1 illustrates one mechanical lysis method 100 that achieves lysis of cells 192 by rapid mixing of a liquid sample 190 that contains both cells 192 and beads 172. In method 100, liquid sample 190 is held in a container 170, and a mixing head 110 is at least partly immersed in liquid sample 190 and spun as indicated by direction 118 (or in the direction opposite to direction 118).

Rapid spinning of mixing head 110 in liquid sample 190 causes lysis of at least some of cells 192 to release at least part of their cell content 194 into liquid sample 190. This lysis may take place through one or more of several different mechanisms. For example, the spinning of mixing head 110 may cause collisions between beads 172 and cells 192, resulting in lysis of cells 192 (as shown in region 150 of FIG. 1). While rapidly spinning in liquid sample 190, mixing head 110 may lyse cells 192 directly hit by mixing head 110 (as shown in region 160 of FIG. 1). The presence of beads 172 in liquid sample 190 may increase the viscosity of liquid sample 190, such that liquid sample 190 moves significantly slower than mixing head 110. This velocity discrepancy may increase the rate of collisions between mixing head 110 and cells 192 as well as increase the impact speed in such collisions, as compared to a liquid sample 190 without beads 172.

The lysis efficiency achieved in method 100 depends on several factors, such as the spin rate, size, and shape of mixing head 110, the size and shape of container 170, the amount and/or size of beads 172 in liquid sample 190, and the viscosity of liquid sample 190. Herein, lysis efficiency is defined as according to the below equation.

$$Lysis Efficiency = 1 - \frac{\# of cells counted after lysis}{\# of cells counted before lysis}$$

In certain implementations, the lysis efficiency increases with the spin rate of mixing head 110, such that a desired lysis efficiency is achieved only if the spin rate exceeds a threshold rate. Thus, in one embodiment, method 100 includes preventing mixing head 110 from spinning at a spin rate that is less than the threshold rate associated with the desired lysis efficiency. In this embodiment, mixing head 110 will not be allowed to spin at a spin rate lower than the threshold rate. This embodiment prevents the undesirable scenario wherein mixing head 110 spins too slowly to achieve the desired lysis efficiency. The difference between the required spin rate and a spin rate that is too slow may be imperceptible to an operator observing mixing head 110 spin in liquid sample 190. In a worst case example of this undesirable scenario, an operator may proceed under the assumption that lysis was effective, and get a false negative result when subsequently testing liquid sample 190 for a substance. Such a false negative may, for example, lead to the wrong and dangerous conclusion that toxic water is safe to drink. The embodiment of method 100 that prevents mixing head 110 from spinning at a spin rate below the threshold rate associated with the desired lysis efficiency also prevents such false negatives.

In the example depicted in FIG. 1, mixing head 110 has two wire loops 112. However, mixing head 110 may be configured differently without departing from the scope hereof. For example, mixing head 110 may have (a) more or fewer than two wire loops 112, (b) a bent helical coil (bent to spiral about a curved axis), and/or (c) one or more flat or curved blades. Similarly, the shape of container 170 may be different from that shown in FIG. 1. Furthermore, it is understood that cells 192 and beads 172 are not drawn to scale.

Cells 192 are, for example, blood cells or bacterial cells. Liquid sample 190 may include a water sample, a blood sample, or a sample of a drink or food intended for human consumption.

In one implementation of method 100, the volume of container 170 is in the range between 0.5 milliliters and 50 milliliters, for example in the range between 1 and 10 milliliters. The volume of liquid sample 190 is less than the volume of container 170, at least to accommodate mixing head 110 inside container 170 and, in some scenarios, also to allow more movement of liquid sample 190 while mixing head 110 is spinning. Container 170 may be generally cylindrical, as shown in FIG. 1, or have a different shape, for example to encourage collisions between cells 192, beads 172, and mixing head 110. In one implementation, at least a portion of container 170 is cylindrical with the cylinder axis substantially coinciding with the rotation axis of mixing head 110. The lateral extent of mixing head 110 (dimensions orthogonal to its rotation axis) are generally less than shortest lateral dimension of container 170 such that mixing head 110 is free to rotate in container 170. It is understood that mixing head 110 may be somewhat flexible, in which case the lateral extent of mixing head 110 may exceed the shortest lateral dimension of container 170. However, contact between mixing head 110 and container 170 may resist spinning of mixing head 110 and either increase the power requirements for mixing head 110 or reduce the spin rate achievable.

In one embodiment of method 100, the spin rate is in the range between 50 Hz and 250 Hz, for example in the range between 100 Hz and 150 Hz. Such spin rates may result in a lysis efficiency greater than 90%, for example approximately 95%. Beads 172 may be rigid beads made of glass, zirconium, ceramic, steel, sand, latex, plastic, or a combination thereof. Beads 172 may be characterized by a diameter in the range between 1 micron and 5 millimeters. In certain scenarios, a bead diameter in the range between 10 microns and 1 millimeter has proven most effective for lysis. In any given liquid sample 190, all beads 172 may have substantially similar diameters. Alternatively, liquid sample 190 may contain a mix of beads 172 having different sizes.

In an embodiment of method 100, the volume of liquid sample 190 is in the range between 1 and 10 milliliters, and the total mass of beads 172 in liquid sample 190 is in the range between 1 and 50 grams. A lower total bead mass may be insufficient to achieve the desired lysis efficiency (e.g., 95%), and a higher total bead mass may preclude mixing head 110 from spinning at the spin rate required for effective lysis (e.g., 95% lysis efficiency).

Without departing from the scope hereof, liquid sample 190 may further include a chemical lysis reagent to aid lysis. The chemical lysis reagent may include surfactant, detergent, an acid, a base, or a combination thereof.

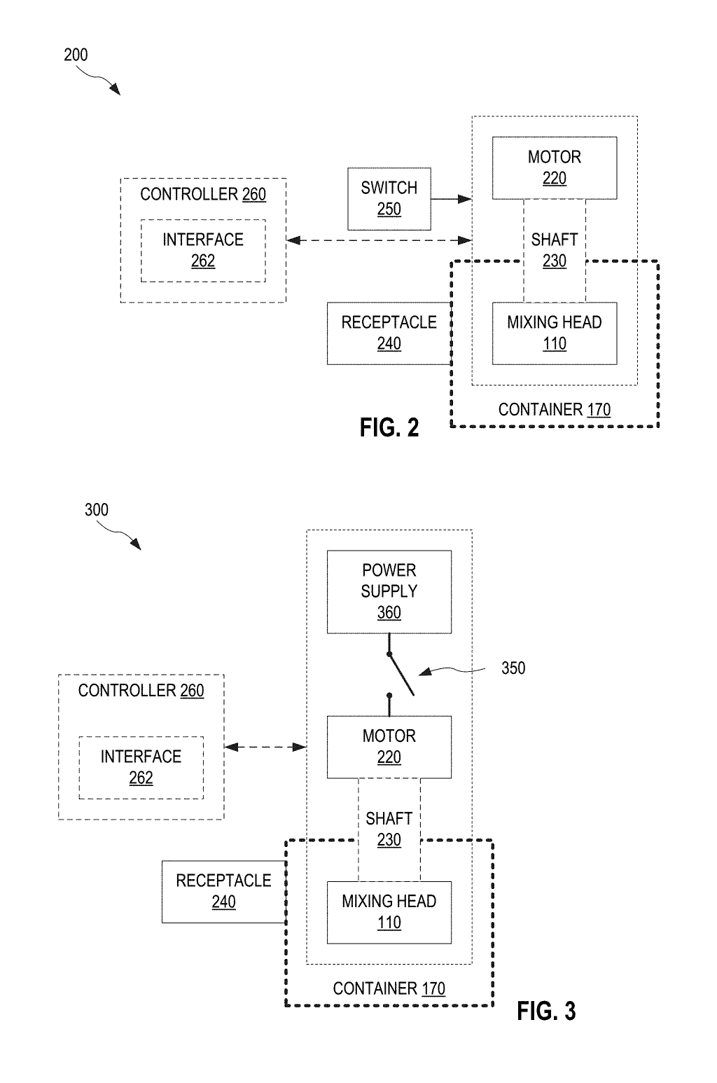

FIG. 2 is a block diagram of one spin-rate controlled mechanical lysis apparatus 200. Apparatus 200 is configured to perform an embodiment of method 100 that prevents the mixing head from spinning at a spin rate that is lower than a predefined threshold rate. Apparatus 200 includes mixing head 110, a motor 220 configured to actuate mixing head 110, a switch 250 preventing spinning of mixing head 110 at a spin rate that is lower than the threshold rate, and a receptacle 240. Receptacle 240 is configured to receive container 170 holding a liquid sample 190 to be lysed by apparatus 200. Apparatus 200 may be provided with out without one or more containers 170.

Motor 220 is mechanically coupled to mixing head 110. Motor 220, mixing head 110, and receptacle 240 are positioned such that, during operation of apparatus 200, mixing head 110 is inside container 170 and motor 220 is outside container 170. In one embodiment, motor 220 is coupled to mixing head 110 via a shaft 230 configured to pass through an opening of container 170.

Switch 250 prevents mixing head 110 from spinning at a spin rate that is lower than the threshold rate. If motor 220 cannot maintain a spin rate of mixing head 110 that exceeds the threshold rate, switch 250 turns off motor 220 or decouples motor 220 from mixing head 110.

Although not explicitly shown in FIG. 2, container 170 may be sealed during operation of apparatus 200. In one embodiment, receptacle 240 seals container 170. In another embodiment, container 170 is sealed by a lid, wherein the lid may have a port for shaft 230. When lysing cells that potentially contain hazardous materials, such as cyanobacterial toxins, there is some risk to the test operator of inhaling aerosols created by mechanical lysis methods. Embodiments of apparatus 200 configured to seal container 170 or compatible with use of a sealed container 170 prevent or at least significantly reduce escape of aerosols from container 170 during operation of apparatus 200.

Although it is possible to use magnetic coupling between motor 220 and mixing head 110, mechanical coupling between motor 220 and mixing head 110 ensures that mixing head 110 spins at the rate imparted by motor 220. In particular due to the presence of beads 172, liquid sample 190 may have high viscosity. In some scenarios, it may be impractical to use magnetic coupling since the viscosity may cause mixing head 110 to slip relative to motor 220 and hence spin at a spin rate that is less than the threshold rate.

Apparatus 200 may include a controller 260 that controls at least certain aspects of the operation of apparatus 200. In one embodiment, controller 260 controls the duration of spinning of mixing head 110. Controller 260 may include an interface 262 that allows an operator to start spinning of mixing head 110, such as a start button. In one implementation, interface 262 also allows the operator to stop spinning of mixing head 110. In another implementation, controller 260 is configured to spin mixing head 110 for a predefined amount of time before automatically turning off. Interface 262 may further include a visual indicator configured to communicate an alert if motor 220 cannot maintain a spin rate of mixing head 110 that exceeds the threshold rate. For battery powered implementations of apparatus 200, this alert may indicate that the battery is in need of replacement. Herein, a “battery” may refer to a single battery or a group of batteries.

FIG. 3 is a block diagram of a spin-rate controlled mechanical lysis apparatus 300 implementing a switch between a motor actuating the mixing head and a power supply powering the motor. Apparatus 300 is an embodiment of apparatus 200 that includes (a) an electric power supply 360 configured to power motor 220, and (b) a switch 350 between electric power supply 360 and motor 220. Switch 350 is an embodiment of switch 250.

Electric power supply 360 may include a battery, a circuit that receives electric power from an external source, or an alternative energy source such as a solar panel.

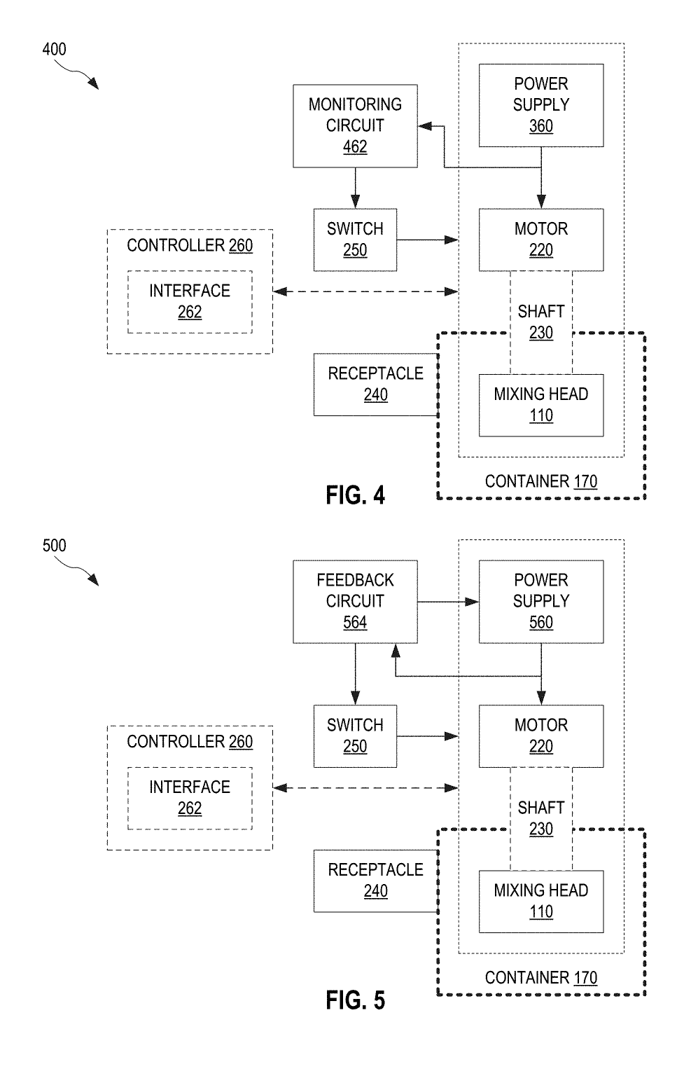

FIG. 4 is a block diagram of a spin-rate controlled mechanical lysis apparatus 400 configured with a monitoring circuit that monitors the level of electric power provided to a motor actuating the mixing head. Apparatus 400 is an embodiment of apparatus 200 that includes (a) electric power supply 360 configured to power motor 220, and (b) a monitoring circuit 462 coupled to switch 250. In operation, monitoring circuit 462 monitors the level of electric power provided by electric power supply 360 to motor 220. If the level of electric power is below a threshold power that corresponds to the threshold rate of spinning of mixing head 110, monitoring circuit 462 causes switch 250 to prevent mixing head 110 from spinning. Apparatus 400 may implement switch 350 as switch 250. In embodiments where electric power supply 360 is a battery, monitoring circuit 462 may monitor the battery voltage.

In one embodiment, apparatus 400 implements a battery in electric power supply 360. When this battery is not able to provide a level of electric power that exceeds the threshold power, switch 250 prevents mixing head 110 from spinning. Commonly, the level of electric power outputted by a battery decreases as the energy stored in the battery diminishes, for example when the battery is nearly drained. In the absence of switch 250, this decreased level of electric power may still be sufficient to make mixing head 110 spin, albeit at a reduced spin rate. Monitoring circuit 462 monitors the level of electric power outputted by the battery to ensure that switch 250 prevents spinning of mixing head 110 when the level of electric power provided by the battery is below the threshold power.

In an alternative embodiment, wherein motor 220 is an alternating current (AC) motor and electric power supply 360 is an AC power supply, the spin rate of motor 220 may be a function of the frequency of AC electric power provided by electric power supply 360. In this alternative embodiment, monitoring circuit 462 may monitor the frequency of the electric power outputted by electric power supply 360 to determine if the frequency corresponds to a spin rate that exceeds the threshold rate. In this alternative embodiment, monitoring circuit 462 may also monitor the AC voltage of the electric power.

FIG. 5 is a block diagram of a spin-rate controlled mechanical lysis apparatus 500 configured with a feedback circuit that adjusts the spin rate of the mixing head based upon an electric power level measurement. Apparatus 500 is an embodiment of apparatus 200 that includes (a) an adjustable electric power supply 560 configured to power motor 220, and (b) a feedback circuit 564 coupled to switch 250 and electric power supply 560. Electric power supply 560 is an adjustable embodiment of electric power supply 360. In operation, feedback circuit 564 monitors the electric power provided by electric power supply 560 to motor 220, and adjusts the electric power as needed to maintain a level and/or frequency of the electric power corresponding to a spin rate of mixing head 110 that exceeds the threshold rate. Feedback circuit 564 may be configured to maintain a constant level and/or frequency of electric power outputted by electric power supply 560. In the event that it is not possible to maintain a level and/or frequency of the electric power corresponding to a spin rate in excess of the threshold rate, feedback circuit 564 causes switch 250 to prevent mixing head 110 from spinning. Apparatus 500 may implement switch 350 as switch 250.

In one embodiment, adjustable electric power supply 560 is a fixed-voltage direct-current (DC) power supply coupled with a voltage regulator, and motor 220 is a DC motor. In this embodiment, the fixed-voltage DC power supply may be (a) a circuit that receives electric power from an external source, e.g., an AC-to-DC converter configured to receive AC power from a grid, (b) a battery, or (c) an alternative energy source such as a solar panel coupled with a circuit that outputs an adjustable fraction of electric power provided by the alternative energy source. The voltage regulator may be an adjustable voltage divider, a linear regulator, or a switching power converter. In operation of this embodiment, feedback circuit 564 monitors the level (e.g., voltage level) of electric power provided by electric power supply 560 to motor 220. If the level of electric power is about to drop below the threshold power that corresponds to the threshold rate of spinning of mixing head 110, feedback circuit 564 adjusts the voltage regulator of electric power supply 560 to increase the level of electric power outputted by electric power supply 560. Feedback circuit 564 may be configured to maintain a constant level of electric power (e.g., constant voltage) outputted by electric power supply 560. In the event that it is not possible to maintain a level of electric power that exceeds the threshold power, feedback circuit 564 causes switch 250 to prevent mixing head 110 from spinning. In embodiments where electric power supply 560 includes a battery and an adjustable voltage divider, feedback circuit 564 may monitor the battery voltage or the voltage output to motor 220 by the voltage regulator, and adjust the voltage regulator as needed to maintain a voltage output to motor 220 that corresponds to a spin rate in excess of the threshold rate.

In another embodiment, adjustable electric power supply 560 is a variable-frequency AC power supply, and motor 220 is an AC motor whose spin rate is a function of the frequency of AC power supplied thereto by electric power supply 560. In operation of this embodiment, feedback circuit 564 monitors the frequency of the electric power outputted by electric power supply 560 to determine if the frequency corresponds to a spin rate of mixing head 110 that exceeds the threshold rate. If needed to maintain a spin rate of mixing head 110 that exceeds the threshold rate (for example a constant spin rate), feedback circuit 564 adjusts the frequency of the AC power outputted to motor 220 by electric power supply 560. Feedback circuit 564 may also monitor the AC voltage of the electric power and make adjustments as needed to maintain a certain spin rate of mixing head 110.

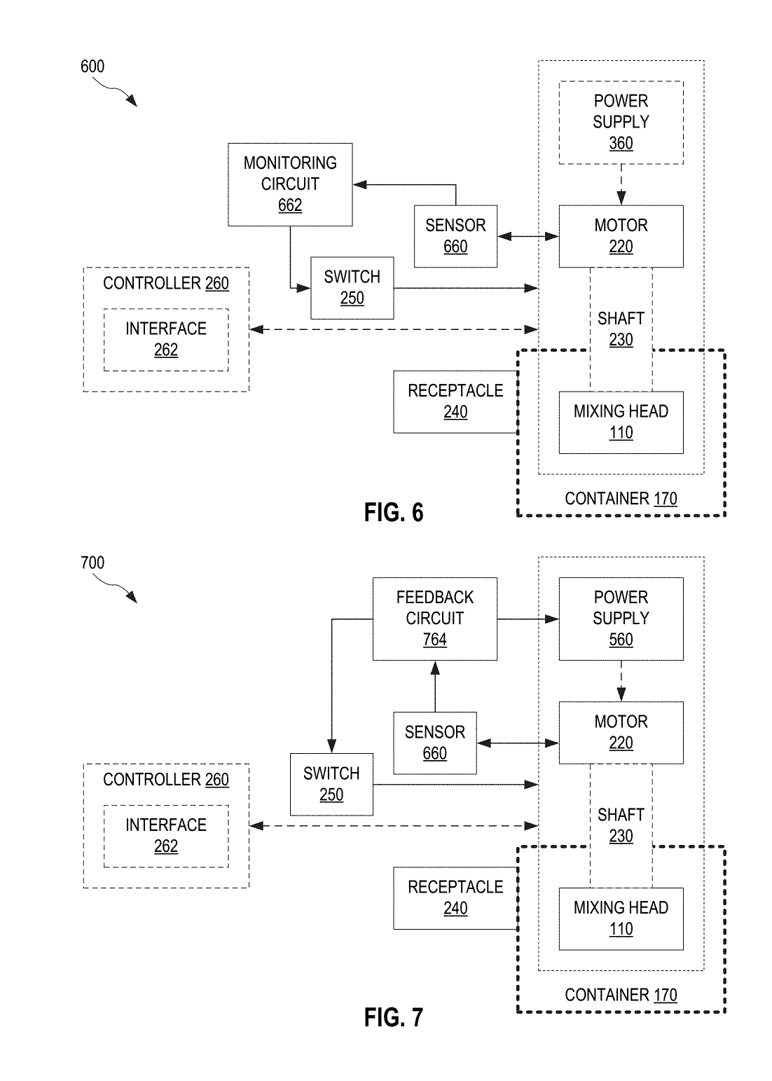

FIG. 6 is a block diagram of a spin-rate controlled mechanical lysis apparatus 600 configured to monitor the spin rate of the mixing head. Apparatus 600 is an embodiment of apparatus 200 that achieves benefits similar to that of apparatus 400. Apparatus 600 includes (a) a sensor 660 that senses the spin rate of mixing head 110, and (b) a monitoring circuit 662 coupled to sensor 660 and switch 250. Apparatus 600 may further include electric power supply 360. Sensor 660 outputs the spin rate, or a parameter indicative of the spin rate, to monitoring circuit 662. Monitoring circuit 662 is similar to monitoring circuit 462 except for monitoring an output of sensor 660 instead of monitoring the level and/or frequency of electric power provided by electric power supply 360. Apparatus 600 may implement switch 350 as switch 250.

FIG. 7 is a block diagram of a spin-rate controlled mechanical lysis apparatus 700 configured with a feedback circuit that adjusts the spin rate of the mixing head based upon a spin rate measurement. Apparatus 700 is an embodiment of apparatus 200 that achieves benefits similar to that of apparatus 500. Apparatus 700 includes (a) adjustable electric power supply 560 configured to power motor 220, (b) sensor 660, and (c) a feedback circuit 764 coupled to sensor 660, electric power supply 560, and switch 250. In operation, feedback circuit 764 monitors the spin rate, or output indicative thereof, provided by sensor 660. If the spin rate is about to drop below the threshold rate, feedback circuit 764 increases the level of electric power, and/or frequency of electric power, outputted by electric power supply 560. Feedback circuit 764 may be configured to maintain a constant spin rate. In the event that it is not possible to maintain a spin rate that exceeds the threshold rate, feedback circuit 764 causes switch 250 to prevent mixing head 110 from spinning. Apparatus 700 may implement switch 350 as switch 250.

In each of apparatuses 600 and 700, sensor 660 may be coupled to motor 220 to sense the spin rate of motor 220 (as depicted in FIGS. 6 and 7). However, without departing from the scope hereof, sensor 660 may instead be coupled to mixing head 110 or shaft 230 to sense their respective spin rate. It is understood that motor 220 may be coupled to mixing head 110 via a gear that steps up or steps down the spin rate of mixing head 110 relative to the spin rate of motor 220, such that the spin rates of motor 220 and mixing head 110 may be different.

Each of apparatuses 200, 300, 400, 500, 600, and 700 may be provided as part of a mechanical lysis system that further includes one or more containers 170. Each container 170 may be preloaded with beads 172 such that a user only needs to add a liquid with cells 192 to be lysed.

A set of containers 170 preloaded with beads 172 may be provided, separately from a mechanical lysis apparatus, as a kit to be used with a mechanical lysis apparatus such as anyone of apparatuses 200, 300, 400, 500, 600, and 700. This kit may further include one or more cleaning containers 170 holding a cleaning solution such as water or bleach (or configured to receive a cleaning solution). Mixing head 110 may be cleaned by running the lysis apparatus with a cleaning container 170 placed in receptacle 240. Cleaning of mixing head 110 may require running the lysis apparatus several times, each time with a different cleaning container 170 and, in certain protocols, with at least some of cleaning containers 170 containing a different type of cleaning liquid than the rest of cleaning containers 170.

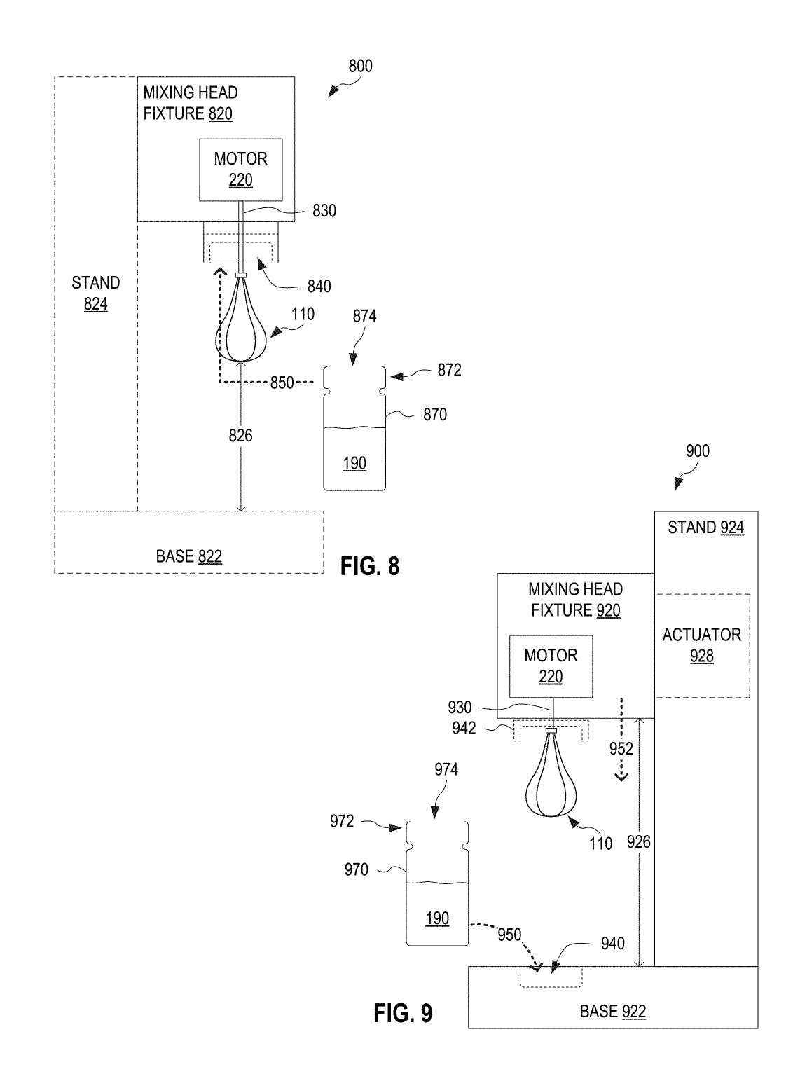

FIG. 8 is a side view of one structural configuration 800 of a mechanical lysis apparatus. Any one of apparatuses 200, 300, 400, 500, 600, 700 may be implemented according to structural configuration 800. In structural configuration 800, motor 220 is implemented in a mixing head fixture 820, a receptacle 840 is mounted beneath mixing head fixture 820, and mixing head 110 is mounted from mixing head fixture 820 via a shaft 830 that extends downwards from mixing head fixture 820 at least to within receptacle 840. Receptacle 840 is configured to hold a top portion 872 of a container 870. When receptacle 840 holds top portion 872, mixing head 110 is inside container 870. Container 870 is an embodiment of container 170. Without departing from the scope hereof, receptacle 840 may be a recess in mixing head fixture 820.

Container 870 has an opening 874 that allows mixing head 110 to enter container 870 when container 870 is placed in receptacle 840. Receptacle 840 may be configured to seal opening 874, for example to prevent liquids or aerosols from escaping container 870 when mixing head 110 spins in liquid sample 190 inside container 870.

Mixing head fixture 820 may be coupled to a base 822 via a stand 824. In one implementation, mixing head fixture 820 is permanently affixed to base 822 (via stand 824) with the distance 826 between mixing head 110 and base 822 sufficient to allow inserting container 170 as schematically indicated by arrow 850. In another implementation, mixing head fixture 820 is removable from base 822 (for example, removable from stand 824), such that container 870 may be mounted in receptacle 840 prior to coupling mixing head fixture 820 to base 822 (for example via stand 824).

FIG. 9 is a side view of another structural configuration 900 of a mechanical lysis apparatus. Any one of apparatuses 200, 300, 400, 500, 600, 700 may be implemented according to structural configuration 900. In structural configuration 900, motor 220 is implemented in a mixing head fixture 920, and mixing head 110 is mounted from mixing head fixture 920 via a shaft 930 that extends downwards from mixing head fixture 920. Mixing head fixture 920 is mounted to a base 922 via a stand 924. The distance 926 between mixing head fixture 920 and base 922 is adjustable. Base 922 forms a recess 940. In a use scenario, a container 970 is seated in recess 940 (as indicated by arrow 950), whereafter mixing head fixture 920 is lowered (as indicated by arrow 952) to place mixing head 110 inside container 970. Container 970 is an embodiment of container 170. Container 970 has an opening 974 that allows mixing head 110 to pass into container 970 when mixing head fixture 920 is lowered.

Stand 924 may include an actuator 928 that adjusts distance 926 as needed to position mixing head 110 in container 970 and remove mixing head 110 from container 970. Alternatively, an operator may manually adjust distance 926 as needed.

In an embodiment, configuration 900 includes a lid 942 that surrounds shaft 930. Lid 942 may close opening 974 to seal container 970 while mixing head 110 is inside container 970. In one implementation, lid 942 is a screw-type lid that is free to rotate about shaft 930 such that lid 942 may be screwed onto a top portion 972 of container 970. In another implementation, lid 942 is pressed onto container 970 over opening 974. In this implementation, lid 942 may be rigidly coupled to mixing head fixture 920, or lid 942 may be a recess in mixing head fixture 920.

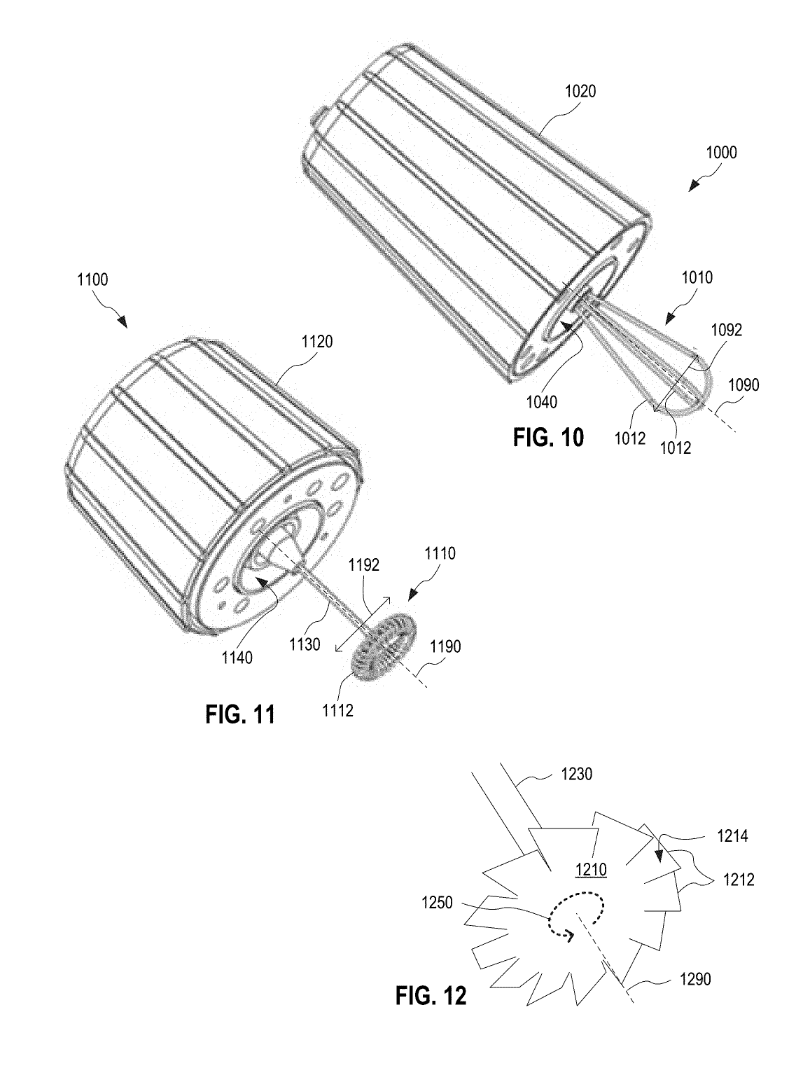

FIG. 10 illustrates one mixing head 1010 that includes two wire loops 1012. Mixing head 1010 is an embodiment of mixing head 110. Mixing head 1010 is configured to spin about an axis 1090. Each wire loop 1012 may be made of metal, e.g., steel, or another material that is compatible with liquid sample 190 and, in certain implementations, also compatible with cleaning of mixing head 1010 using a cleaning solution. Without departing from the scope hereof, mixing head 1010 may include more than two wire loops 1012 or have only a single wire loop 1012. Also without departing from the scope hereof, the shape of wire loops 1012 may be different from that depicted in FIG. 10.

FIG. 10 shows mixing head 1010 coupled to a mixing head fixture 1020 to form an assembly 1000. Mixing head fixture 1020 is an example of either one of mixing head fixtures 820 and 920. Mixing head fixture 1020 forms a recess 1040 that is an example of either one of receptacle 840 and lid 942. Although not shown in FIG. 10, mixing head 1010 may be coupled to mixing head fixture 1020 via a shaft.

FIG. 11 illustrates one mixing head 1110 that includes a helical coil 1112. Mixing head 1110 is configured to spin about an axis 1190. Helical coil 1112 wraps around axis 1190. Mixing head 1110 is an embodiment of mixing head 110. Helical coil 1112 may be made of the same materials discussed above for wire loops 1012. Without departing from the scope hereof, helical coil 1112 may wrap only partway around axis 1190.

FIG. 11 shows mixing head 1110 coupled to a mixing head fixture 1120, via a shaft 1130, to form an assembly 1100. Mixing head fixture 1120 is an example of either one of mixing head fixtures 820 and 920, and shaft 1130 is an example of shaft 830. Mixing head fixture 1120 forms a recess 1140 that is an example of either one of receptacle 840 and lid 942.

FIG. 12 illustrates one mixing head 1210 with a plurality of blades 1212. Mixing head 1110 is configured to spin about an axis 1290, as indicated by arrow 1250 or in the direction opposite arrow 1250. Mixing head 1110 is an embodiment of mixing head 110. Mixing head 1110 may be made of the same materials discussed above for wire loops 1012. Each blade 1212 is non-perpendicular to axis 1290 such that, when mixing head 1110 is immersed in liquid sample 190 and spins about axis 1290, a surface 1214 of each blade 1212 pushes on liquid sample 190. Without departing from the scope hereof, mixing head 1110 may include more or fewer blades 1212 than depicted in FIG. 12. Mixing head 1110 may be mounted on a shaft 1230.

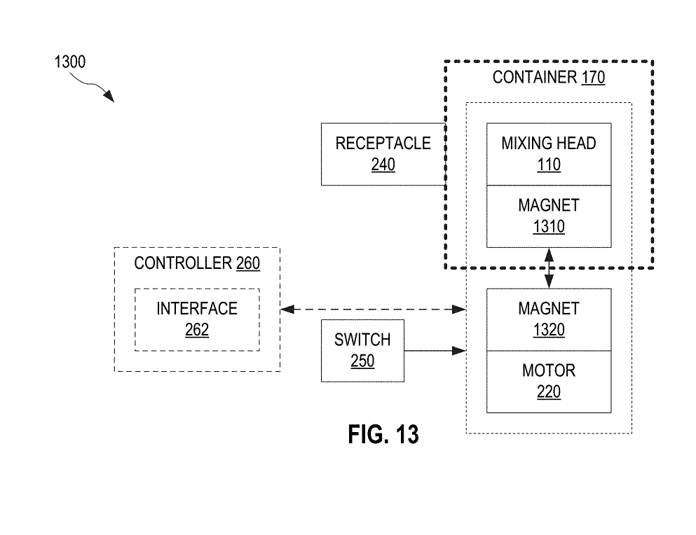

FIG. 13 is a block diagram of one spin-rate controlled mechanical lysis apparatus 1300 that utilizes magnetic coupling between a mixing head and a motor actuating the mixing head. Apparatus 1300 is similar to apparatus 200 apart from utilizing magnetic coupling between mixing head 110 and motor 220. Any one of apparatuses 300, 400, 500, 600, and 700 may be modified to use the magnetic coupling of apparatus 1300.

In apparatus 1300, mixing head 110 is rigidly mounted to a magnet 1310, and motor 220 has a magnet 1320 rigidly mounted thereto. In operation, mixing head 110 is positioned in container 170 together with magnet 1310, and container 170 is positioned such that magnets 1310 and 1320 are magnetically coupled to each other through a wall of container 170 (for example the bottom of container 170). Motor 220 rotates magnet 1320, which causes magnet 1310 and mixing head 110 to rotate as well due to the magnetic coupling between magnets 1310 and 1320.

Although, as discussed above in reference to FIG. 2, the magnetic coupling may “slip” when liquid sample 190 has high viscosity, magnetic coupling, as used by apparatus 1300, may be sufficient for some liquid samples 190. Magnetic coupling has potential advantages. For example, it is possible to fully enclose mixing head 110 and magnet 1310 in a sealed container 170, without having to provide a seal around a shaft between mixing head 110 and motor 220. A standard, off-the-shelf container may be used.

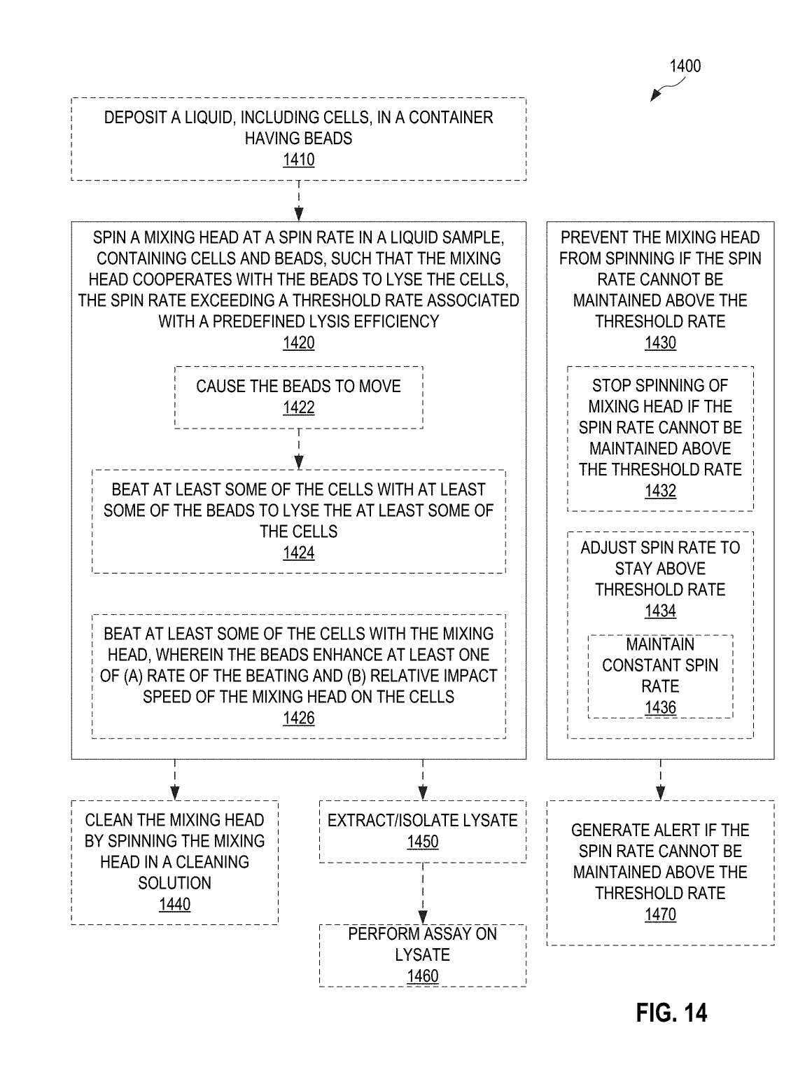

FIG. 14 illustrates one mechanical lysis method 1400. Method 100 is an embodiment of method 1400. Method 1400 includes steps 1420 and 1430. Steps 1420 and 1430 are performed by apparatus 200, 300, 400, 500, 600, 700, or 1300, for example.

Step 1420 spins a mixing head in a liquid sample that contains cells and beads, such that the mixing head cooperates with the beads to lyse the cells, as discussed above in reference to FIG. 1. Step 1420 spins the mixing head at a spin rate that exceeds a threshold rate associated with a predefined lysis efficiency. In one example of step 1420, mixing head 110 spins in liquid sample 190, held in container 170, at a spin rate that exceeds a threshold rate associated with a predefined lysis efficiency, for example 95%. As discussed above in reference to FIG. 1, the threshold rate corresponding to a certain lysis efficiency may depend on several factors. Thus, the threshold rate of step 1420 may be pre-calibrated for a given scenario, such as a certain configuration of the mixing head and container and certain properties of the liquid sample.

As discussed above in reference to FIG. 1, spinning of a mixing head in a liquid sample with cells and beads may result in lysis of beads through several mechanisms. In one scenario, spinning of the mixing head in step 1420 results in steps 1422 and 1424. Step 1422 causes the beads to move, and step 1424 beats at least some of the cells with at least some of the beads to lyse at least some of the cells (as shown in region 150 in FIG. 1). In another scenario, spinning of the mixing head results in step 1426. In step 1426, the mixing head beats at least some of the cells, and the rate and/or relative impact speed of the mixing head on the cells is enhanced by the presence of the beads (see region 152 in FIG. 1). In yet another scenario, spinning of the mixing head results in both steps 1422 and 1424 and step 1426.

Step 1430 prevents the mixing head from spinning in step 1420 if the spin rate cannot be maintained above the threshold rate. In one example of step 1430, switch 250 and/or feedback circuit 564 or 764 prevents mixing head 110 from spinning at a spin rate that is less than the threshold rate, as discussed in further detail above in reference to FIGS. 2-7. Switch 250 may cooperate either with monitoring circuit 462 or 662 or with feedback circuit 564 or 764 to stop spinning of mixing head 110. Step 1430 may include one or both of steps 1432 and 1434. Step 1432 stops the spinning of the mixing head if the spin rate cannot be maintained above the threshold rate. In one example of step 1432, switch 250 stops the spinning of mixing head 110 if the spin rate cannot be maintained above the threshold rate. Step 1434 adjusts the spin rate to stay above the threshold rate. In one example of step 1434, feedback circuit 564 or 764 adjust the level of electric power outputted by electric power supply 560 to adjust the spin rate of mixing head 110. Step 1434 may include a step 1436 of maintaining a constant spin rate. In one example of step 1436, feedback circuit 564 or 764 adjusts the level and/or frequency of electric power outputted by electric power supply 560 to maintain a constant spin rate of mixing head 110.

Method 1400 may further include a step 1410 the precedes step 1420. In step 1410, an operator or an automated sample processing system deposits a liquid, that includes the cells to be lysed, in a container that contains beads, so as to form the liquid sample that is processed in step 1420. In one example of step 1410, an operator or automated sample processing system deposits a liquid, including cells 192, in container 170 preloaded with beads 172.

In certain embodiments, method 1400 includes a step 1470 of generating an alert if the spin rate cannot be maintained above the threshold rate. In one example of step 1470, interface 262 generates an alert if the spin rate of mixing head 110 cannot be maintained above the threshold rate, as discussed in further detail above in reference to FIG. 2.

To avoid carry over of sample contents between processing of different samples using the same mixing head, method 1400 may include a step 1440 of cleaning the mixing head by spinning the mixing head in a cleaning solution. In one example of step 1440, a container 170 holding a cleaning solution, such as water or bleach, is placed in apparatus 200 or 1300, and mixing head 110 is spun in the cleaning solution. Method 1400 may repeat step 1440 with a series of such containers, some of which may contain other types of cleaning solutions than the others.

In one embodiment, method 1400 is extended to also include processing of lysate generated in step 1420. In this embodiment, method 1400 includes one or both of steps 1450 and 1460. Step 1450 extracts or isolates lysate from the liquid sample. The separation method may be chosen according to the volume of lysed sample to be recovered and the requirements for the degree of separation of the plurality of beads and sample. Step 1450 may utilize a variety of separation methods. If the lysate obtained in step 1450 may be completely free of beads, an operator or filtering system may filter the liquid sample. Alternatively, step 1450 may use a centrifuge to separate beads from a bead-free lysate. If complete removal of beads is not necessary, an operator or an automated sample handling system may extract lysed sample from an upper portion of the liquid sample after that at least most of the beads have precipitated to the bottom of the container. Step 1460 performs an assay on the lysate. In one example of step 1460, the lysate extracted or isolated in step 1450 is forwarded to processing in an assay system or device. In another example of step 1460, which does not require removal of the beads, method 1400 bypasses step 1450 and forwards the liquid sample of step 1420 (after lysis) to processing in an assay system or device.

Without departing from the scope hereof, the liquid sample processed in step 1420 may further include a chemical lysis reagent to aid lysis. The chemical lysis reagent may include surfactant, detergent, an acid, a base, or a combination thereof.

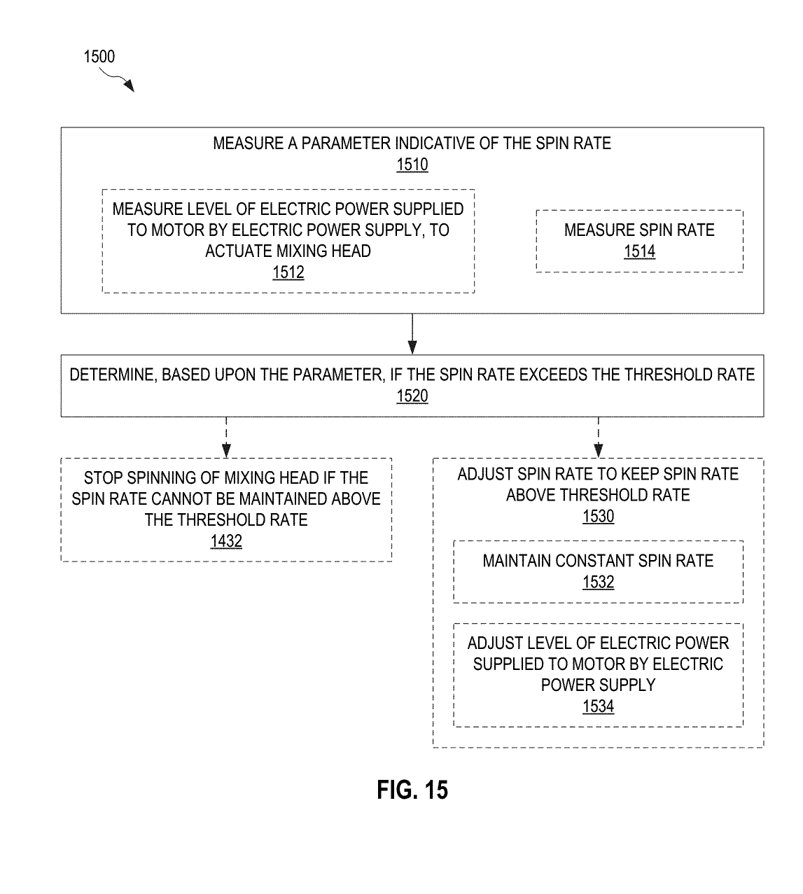

FIG. 15 illustrates one method 1500 for preventing the mixing head in a mechanical lysis apparatus from spinning at a rate that is below a threshold rate associated with a desired lysis efficiency. Method 1500 is an embodiment of step 1430 of method 1400.

In a step 1510, method 1500 measures a parameter that is indicative of the spin rate of the mixing head. Step 1510 may include a step 1512 or a step 1514. Step 1512 measures the level and/or frequency of electric power supplied to a motor that actuates the mixing head. In one example of step 1512, monitoring circuit 462 or feedback circuit 564 senses the level and/or frequency of electric power outputted by electric power supply 360 or 560, respectively, to motor 220. Step 1514 measures the spin rate. In one example of step 1514, sensor 660 senses the spin rate of mixing head 110 and communicates a corresponding output to monitoring circuit 662 or feedback circuit 764, as discussed in further detail above in reference to FIGS. 6 and 7.

In a step 1520, method 1500 determines, based upon the parameter obtained in step 1510, if the spin rate exceeds the threshold spin rate. In one example of step 1520, monitoring circuit 462 or feedback circuit 564 determines if the level and/or frequency of electric power, measured in step 1512, is above a threshold power or frequency, respectively, that corresponds to the threshold rate. In another example of step 1520, monitoring circuit 662 or feedback circuit 764 determines if the spin rate, measured in step 1514, exceeds the threshold rate. It is understood that monitoring circuit 662 and feedback circuit 764 may make this determination based upon an actual spin rate received from sensor 660 or based upon an output of sensor 660 that is indicative of the actual spin rate.

Method 1500 may further include one or both of steps 1432 and 1530. Step 1530 adjusts the spin rate to keep the spin rate above the threshold rate. In one example of step 1530, feedback circuit 564 or 764 adjusts the spin rate of mixing head 110 to keep the spin rate above the threshold rate. Step 1530 may include a step 1532 of maintaining a constant spin rate. In one example of step 1532, feedback circuit 564 or 764 adjusts the spin rate of mixing head 110 to maintain a constant spin rate that exceeds the threshold rate. In one implementation, step 1530 includes a step 1534 of adjusting the level and/or frequency of electric power supplied by an electric power supply to a motor actuating the mixing head. In one example of step 1534, feedback circuit 564 adjusts the level of electric power supplied to motor 220 by electric power supply 560.

### EXAMPLE 1

### Lysis Efficiency as Function of Spin Rate for Helical-Coil Mixing Head

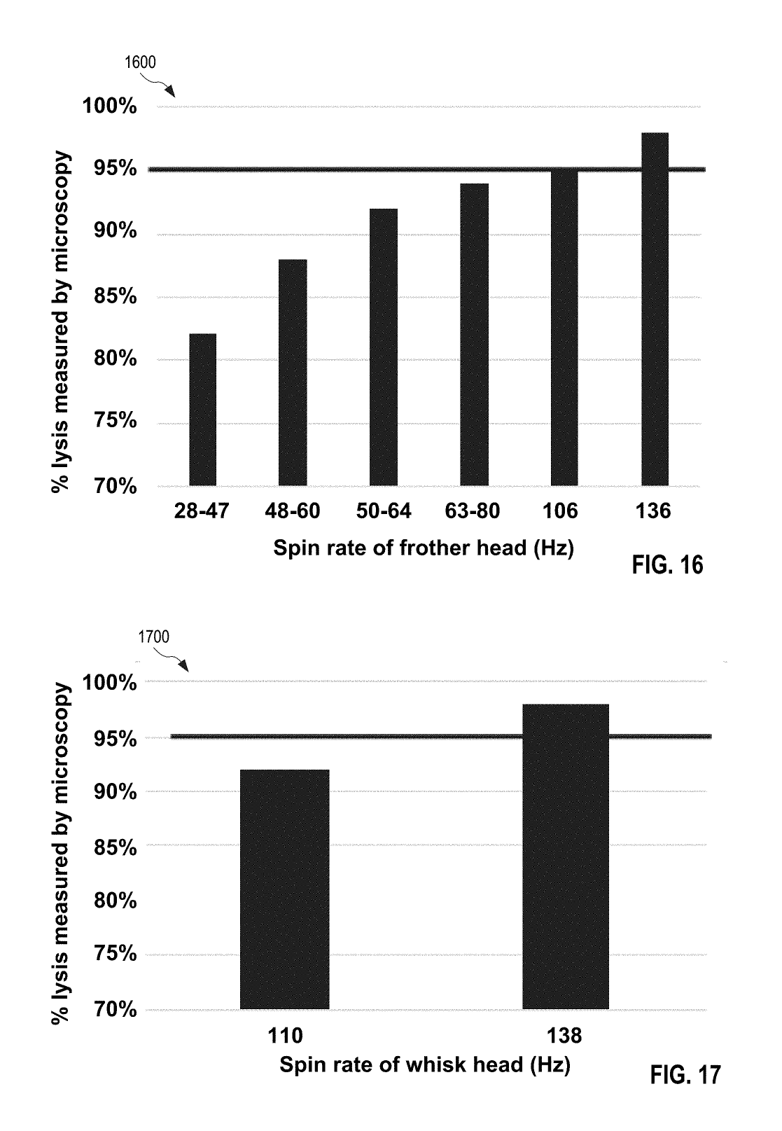

FIG. 16 is a data plot 1600 showing example lysis data achieved in mechanical lysis using a helical-coil mixing head similar to mixing head 1110 of FIG. 11. Data plot 1600 shows the lysis efficiency, as measured by microscope, as a function of the spin rate of the mixing head, for a 5 milliliter water sample containing the LE3 strand of Microcystis aeruginosa (a type of cyanobacteria). The data of FIG. 16 was collected using a container 170 having a diameter of 34 millimeters and a height of approximately 70 millimeters. The outer diameter of the helical-coil mixing head (see dimension 1192 in FIG. 11) was approximately 23 millimeters. The water sample was loaded into the container after preloading the container with 6.5 grams of silica beads. The silica beads were characterized by a diameter of approximately 100 microns. It is evident that the lysis efficiency increases with spin rate. Data plot 1600 illustrates the significance of maintaining a sufficiently high spin rate to achieve a desired lysis efficiency. For example, data plot 1600 shows that if the desired lysis efficiency is 95%, it is necessary to maintain a spin rate that exceeds approximately 105 Hz. Data plot 1600 was recorded using a battery powered mixing head, and the spin rate was regulated by pulse width modulation of the battery power output delivered to the mixing head. It was also found that the battery, when nearly drained, was able to spin the mixing head but not at the spin rate required to achieve 95% lysis efficiency.

### EXAMPLE 2

### Lysis Efficiency as Function of Spin Rate for Wire-Loop Mixing Head

FIG. 17 is a data plot 1700 showing example lysis data achieved in mechanical lysis using a wire-loop mixing head similar to mixing head 1010 of FIG. 10. Data plot 1700 shows the lysis efficiency, as measured by microscope, for two different spin rates of the mixing head, for a 5 milliliter water sample containing the LE3 strand of Microcystis aeruginosa (a type of cyanobacteria). The data of data plot 1700 was collected under the same conditions as the data of data plot 1600, apart from using a different mixing head. The outer diameter of the wire-loop mixing head (see dimension 1092 in FIG. 10) was approximately 23 millimeters. Data plot 1700 underlines the significance of maintaining a sufficiently high spin rate to achieve a desired lysis efficiency. For example, data plot 1700 shows that if the desired lysis efficiency is 95%, a spin rate of 138 Hz is sufficient, but a spin rate of 110 Hz is not sufficient. Also here it was found that the battery, when nearly drained, was able to spin the mixing head but not at the spin rate required to achieve 95% lysis efficiency.

### EXAMPLE 3

### Lysis Efficiency as Function of Spin Rate for Bladed Mixing Head

An experiment similar to those of Examples 1 and 2 was conducted using a bladed mixing head similar to that of FIG. 12. At a spin rate of 133 Hz, the bladed mixing head produced a lysis efficiency of only 79%, which is significantly less than that obtained with the helical-coil mixing head and the wire-loop mixing head.

### EXAMPLE 4

### Lysis Efficiency as Function of Amount of Beads for Helical-Coil Mixing Head

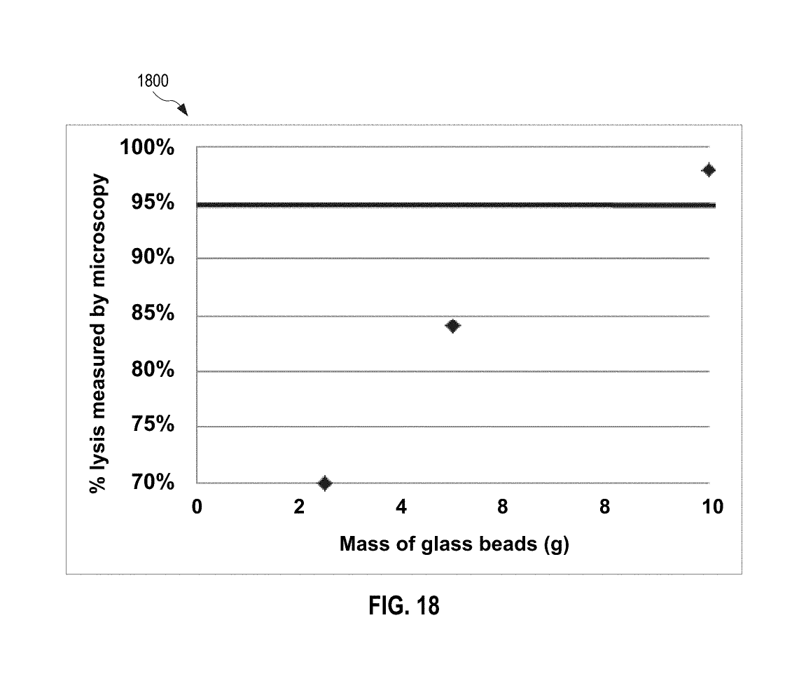

FIG. 18 is a data plot 1800 showing example lysis data achieved in mechanical lysis using the helical-coil mixing head of Example 1. Data plot 1800 shows the lysis efficiency, as measured by microscope, for different amounts of silica beads (indicated by the total mass of the silica beads). In Example 4, the 5 milliliter water sample contained the LB2063 strand of Microcystis aeruginosa (obtained from UTEX Culture Collection of Algae at the University of Texas at Austin). The mixing head was operated at a spin rate of 136 Hz. Data plot 1800 shows that the lysis efficiency increases with the amount of beads, at least within the tested range of total bead mass.

Combinations of Features

Features described above as well as those claimed below may be combined in various ways without departing from the scope hereof. For example, it will be appreciated that aspects of one mechanical lysis method or apparatus, described herein may incorporate or swap features of another mechanical lysis method or apparatus, described herein. The following examples illustrate possible, non-limiting combinations of embodiments described above. It should be clear that many other changes and modifications may be made to the systems and methods described herein without departing from the spirit and scope of this invention:

(A1) One example mechanical lysis method includes spinning a mixing head at a spin rate in a liquid sample, containing cells and beads, such that the mixing head cooperates with the beads to lyse the cells, wherein the spin rate exceeds a threshold rate associated with a predefined lysis efficiency. This mechanical lysis method further includes preventing the mixing head from spinning if the spin rate cannot be maintained above the threshold rate.

(A2) In the mechanical lysis method denoted as (A1), the step of spinning may include causing the beads to move, and beating at least some of the cells with at least some of the beads to lyse the at least some of the cells.

(A3) In either of the mechanical lysis methods denoted as (A1) and (A2), the step of spinning may include beating at least some of the cells with the mixing head, wherein the beads enhance at least one of (a) rate of the beating and (b) relative impact speed of the mixing head on the cells.

(A4) Any of the mechanical lysis methods denoted as (A1) through (A3) may include (a) in the step of spinning, include spinning the mixing head inside a container holding the liquid sample, and (b) actuating the mixing head with a motor disposed outside the container and mechanically coupled to the mixing head through a top of the container.

(A5) Any of the mechanical lysis methods denoted as (A1) through (A4) may further include generating an alert if the spin rate cannot be maintained above the threshold rate.

(A6) Any of the mechanical lysis methods denoted as (A1) through (A5) may further include measuring a parameter indicative of the spin rate, and determining, based upon the parameter, if the spin rate exceeds the threshold rate.

(A7) The mechanical lysis method denoted as (A6) may further include using a motor to actuate the mixing head, and powering the motor with an electric power supply, wherein the parameter is at least one of (i) a power level of electric power supplied to the motor by the electric power supply and (ii) a frequency of the electric power.

(A8) The mechanical lysis method denoted as (A7) may further include adjusting at least one of the power level and the frequency to keep the spin rate above the threshold rate.

(A9) Any of the mechanical lysis methods denoted as (A6) through (A8) may further include adjusting, based upon the parameter, the spin rate to keep the spin rate above the threshold rate.

(A10) Any of the mechanical lysis methods denoted as (A6) through (A8) may further include adjusting, based upon the parameter, the spin rate to maintain the spin rate at a constant rate that exceeds the threshold rate.

(A11) In the mechanical lysis method denoted as (A6), the parameter may be the spin rate or an output from a spin rate sensor.

(A12) Any of the mechanical lysis methods denoted as (A1) through (A11) may further include depositing a liquid, including the cells, in a container having the beads, to form the liquid sample.

(A13) Any of the mechanical lysis methods denoted as (A1) through (A12) may further include, after the step of spinning, cleaning the mixing head by spinning the mixing head in a cleaning solution.

(B1) One example lysis apparatus includes (a) a receptacle for holding a container containing a liquid sample with cells and beads, (b) a mixing head configured to spin in the liquid sample, (c) a motor configured to spin the mixing head such that the mixing head cooperates with the beads to lyse the cells, and (d) a switch configured to prevent the mixing head from spinning at a spin rate lower than a threshold rate associated with a predefined lysis efficiency.

(B2) In the lysis apparatus denoted as (B1), the switch may be coupled to the motor and configured to turn off the motor when the motor cannot maintain a spin rate of the mixing head that exceeds the threshold rate.

(B3) Either of the lysis apparatuses denoted as (B1) and (B2) may further include a visual indicator for communicating an alert when the motor cannot maintain a spin rate of that exceeds the threshold spin rate.

(B4) Any of the lysis apparatuses denoted as (B1) through (B3) may further included a monitoring circuit coupled with the switch and configured to (1) monitor a parameter indicative of the spin rate, (2) based upon the parameter, determine if the spin rate exceeds the threshold rate, and (3) control the switch to prevent spinning of the mixing head if the spin rate does not exceed the threshold rate.

(B5) In the lysis apparatus denoted as (B4), the monitoring circuit may be configured to (i) measure a power level associated with the motor, and (ii) compare the power level to a threshold power associated with the threshold rate to determine if the spin rate exceeds the threshold rate.

(B6) The lysis apparatus denoted as (B5) may further include an electric power supply for powering the motor, and the power level measured by the monitoring circuit may be a level of electric power supplied to the motor by the electric power supply.

(B7) In the lysis apparatus denoted as (B6), the electric power supply may include a battery.

(B8) The lysis apparatus denoted as (B4) may further include a sensor for measuring spin rate of the mixing head, and the parameter may be an output of the sensor.

(B9) Any of the lysis apparatuses denoted as (B1) through (B3) may further include an electric power supply for powering the motor, and a feedback circuit configured to maintain a constant level of electric power supplied to the motor by the electric power supply during spinning of the mixing head, wherein the constant level is above a threshold power level corresponding to the threshold rate.

(B10) In the lysis apparatus denoted as (B9), the electric power supply may include a battery.

(B11) Any of the lysis apparatuses denoted as (B1) through (B3) may further include (1) a sensor for measuring spin rate of the mixing head, and (2) a feedback circuit coupled between the sensor and the motor, and configured to maintain the spin rate at a constant value above the threshold rate.

(B12) Any of the lysis apparatuses denoted as (B1) through (B11) may further include an interface for receiving a start command from a user, and a controller configured to (I) initiate spinning of the mixing head upon receiving the start command, and (II) stop spinning of the mixing head after a predefined duration.

(B13) In any of the lysis apparatuses denoted as (B1) through (B12), the mixing head may include a plurality of wire loops.

(B14) In any of the lysis apparatuses denoted as (B1) through (B13), the mixing head may include a helical coil spiraling about a curved axis.

(B15) In any of the lysis apparatuses denoted as (B1) through (B14), the receptacle may be configured to hold a top portion of the container and seal a partly open top of the container.

(B16) In any of the lysis apparatuses denoted as (B1) through (B15), the mixing head may be mounted on a shaft that extends downwards to position the mixing head in the container when the container is held by the receptacle.

(B17) In any of the lysis apparatuses denoted as (B1) through (B16), the receptacle may have a recess configured to seat therein a bottom portion of the container, and the mixing head may be mounted above the recess.

(B18) The lysis apparatus denoted as (B17) may further include an actuator configured to adjust distance between the mixing head and the recess to lower the mixing head into the container and raise the mixing head from the container to allow removal of the container from the recess.

Changes may be made in the above systems and methods without departing from the scope hereof. It should thus be noted that the matter contained in the above description and shown in the accompanying drawings should be interpreted as illustrative and not in a limiting sense. The following claims are intended to cover generic and specific features described herein, as well as all statements of the scope of the present systems and methods, which, as a matter of language, might be said to fall therebetween.

What is claimed is:

1. A mechanical lysis method, comprising:
spinning a mixing head at a spin rate in a liquid sample, containing cells and beads, such that the mixing head cooperates with the beads to lyse the cells, the spin rate exceeding a threshold rate associated with a predefined lysis efficiency; and
preventing the mixing head from spinning if the spin rate cannot be maintained above the threshold rate.

2. The mechanical lysis method of claim 1, the step of spinning comprising:
causing the beads to move; and
beating at least some of the cells with at least some of the beads to lyse the at least some of the cells.

3. The mechanical lysis method of claim 1, the step of spinning comprising beating at least some of the cells with the mixing head, the beads enhancing at least one of (a) rate of the beating and (b) relative impact speed of the mixing head on the cells.

4. The mechanical lysis method of claim 1,
the step of spinning comprising spinning the mixing head inside a container holding the liquid sample; and
further comprising actuating the mixing head with a motor disposed outside the container and mechanically coupled to the mixing head through top of the container.

5. The mechanical lysis method of claim 1, further comprising generating an alert if the spin rate cannot be maintained above the threshold rate.

6. The mechanical lysis method of claim 1, further comprising:
measuring a parameter indicative of the spin rate; and
determining, based upon the parameter, if the spin rate exceeds the threshold rate.

7. The mechanical lysis method of claim 6, further comprising using a motor to actuate the mixing head, and powering the motor with an electric power supply, the parameter being at least one of a power level of electric power supplied to the motor by the electric power supply and a frequency of the electric power.

8. The mechanical lysis method of claim 7, further comprising, adjusting at least one of the power level and the frequency to keep the spin rate above the threshold rate.

9. The mechanical lysis method of claim 6, further comprising adjusting, based upon the parameter, the spin rate to keep the spin rate above the threshold rate.

10. The mechanical lysis method of claim 6, further comprising adjusting, based upon the parameter, the spin rate to maintain the spin rate at a constant rate that exceeds the threshold rate.

11. The mechanical lysis method of claim 6, the parameter being the spin rate or an output from a spin rate sensor.

12. The mechanical lysis method of claim 1, further comprising depositing a liquid, including the cells, in a container having the beads, to form the liquid sample.

13. The mechanical lysis method of claim 1, further comprising, after the step of spinning, cleaning the mixing head by spinning the mixing head in a cleaning solution.

14. A lysis apparatus, comprising:
a receptacle for holding a container containing a liquid sample with cells and beads;
a mixing head configured to spin in the liquid sample;
a motor configured to spin the mixing head such that the mixing head cooperates with the beads to lyse the cells; and
a switch configured to prevent the mixing head from spinning at a spin rate lower than a threshold rate associated with a predefined lysis efficiency.

15. The lysis apparatus of claim 14, the switch being coupled to the motor and configured to turn off the motor when the motor cannot maintain a spin rate of the mixing head that exceeds the threshold rate.

16. The lysis apparatus of claim 14, further comprising a visual indicator for communicating an alert when the motor cannot maintain a spin rate of that exceeds the threshold spin rate.

17. The lysis apparatus of claim 14, further comprising a monitoring circuit coupled with the switch and configured to (a) monitor a parameter indicative of the spin rate, (b) based upon the parameter, determine if the spin rate exceeds the threshold rate, and (c) control the switch to prevent spinning of the mixing head if the spin rate does not exceed the threshold rate.

18. The lysis apparatus of claim 17, the monitoring circuit being configured to (a) measure a power level associated with the motor, and (b) compare the power level to a threshold power associated with the threshold rate to determine if the spin rate exceeds the threshold rate.

19. The lysis apparatus of claim 18, further comprising an electric power supply for powering the motor, the power level measured by the monitoring circuit being a level of electric power supplied to the motor by the electric power supply.

20. The lysis apparatus of claim 19, the electric power supply including a battery.

21. The lysis apparatus of claim 17, further comprising a sensor for measuring spin rate of the mixing head, the parameter being an output of the sensor.

22. The lysis apparatus of claim 14, further comprising:
an electric power supply for powering the motor; and
a feedback circuit configured to maintain a constant level of electric power supplied to the motor by the electric power supply during spinning of the mixing head, the constant level being above a threshold power level corresponding to the threshold rate.

23. The lysis apparatus of claim, 22, the electric power supply including a battery.

24. The lysis apparatus of claim 14, further comprising:
a sensor for measuring spin rate of the mixing head; and
a feedback circuit coupled between the sensor and the motor, and configured to maintain the spin rate at a constant value above the threshold rate.

25. The lysis apparatus of claim 14, further comprising:
an interface for receiving a start command from a user; and
a controller configured to (a) initiate spinning of the mixing head upon receiving the start command, and (b) stop spinning of the mixing head after a predefined duration.

26. The lysis apparatus of claim 14, the mixing head including a plurality of wire loops.

27. The lysis apparatus of claim 14, the mixing head including a helical coil spiraling about a curved axis.

28. The lysis apparatus of claim 14, the receptacle being configured to hold a top portion of the container and seal a partly open top of the container.

29. The lysis apparatus of claim 28, the mixing head being mounted on a shaft that extends downwards to position the mixing head in the container when the container is held by the receptacle.

30. The lysis apparatus of claim 14, the receptacle having a recess configured to seat therein a bottom portion of the container, the mixing head being mounted above the recess.

31. The lysis apparatus of claim 30, further comprising an actuator configured to adjust distance between the mixing head and the recess to (a) lower the mixing head into the container, and (b) raise the mixing head from the container to allow removal of the container from the recess.
